(19) United States
(12) Plant Patent Application Publication
(10) Pub. No.: US 2006/0037113 P1
(43) Pub. Date: Feb. 16, 2006

(54) Miniature rose plant 'PACthree'

(50) ROSA HYBRID; MINIATURE ROSE

(76) Inventor: Daniel Jauchen, Santa Barbara, CA (US)

Correspondence Address: Daniel Jauchen, P.O. Box 6354, Santa Barbara, CA, 93111, US

(21) Appl. No.: 10/918,800

(22) Filed: Aug. 16, 2004

(51) Int. Cl. A01H 5/00

(52) U.S. Cl. PLT/122

(57) ABSTRACT

A new miniature rose plant which has abundant, red colored flowers and attractive foliage. The variety successfully propagates from softwood cuttings and is suitable for year round production in commercial glass houses as a flowering pot plant. This new and distinct variety has shown to be uniform and stable in the resulting generations from asexual propagation.

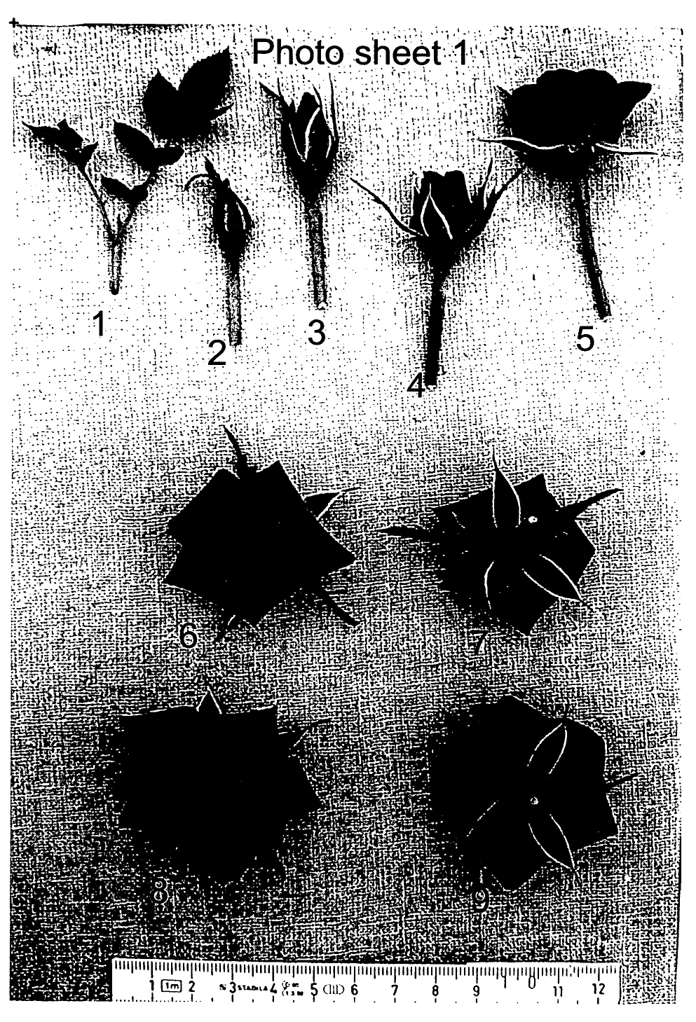

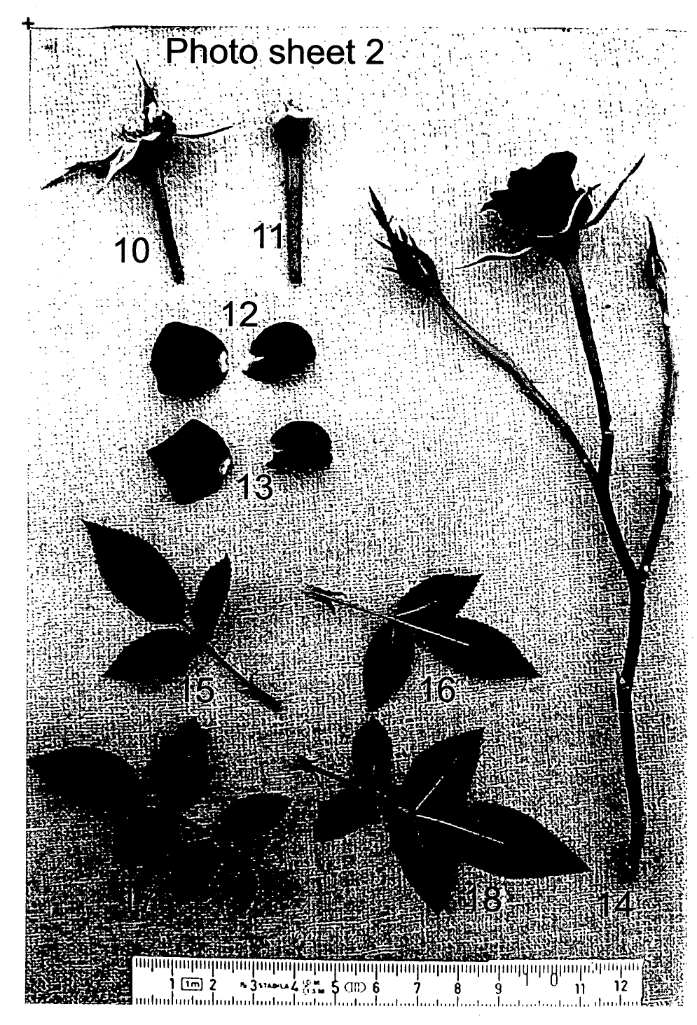

### BOTANICAL DESIGNATION

Rosa hybrida

### Variety Denomination

‘PACthree’.

### BACKGROUND OF THE INVENTION

The present invention constitutes a new and distinct variety of miniature rose plant, which was developed by artificially pollinating an unnamed seedling(unpatented) with an unnamed seedling (unpatented). The two parents were crossed in the summer of 2002 and the resulting seed was sown in December 2002, in a controlled glasshouse environment. Out of these seedlings one seedling was selected, as the new variety and named ‘PACthree’. The new rose may be distinguished from its seed parent, an unnamed seedling, by the following combination of characteristics:

1. ‘PACthree’ has medium double flowers, while the unnamed seedling has small double flowers.

2. ‘PACthree’ has red colored petals, while the unnamed seedling has pink petals.

The new variety may distinguished from its pollen parent, an unnamed seedling created by the same inventor, by the following combination of characteristics:

1. ‘PACthree’ has medium flowers and foliage as compared to the unnamed seedling.

2. ‘PACthree’ has red colored petals, while the unnamed seedling has dark pink petals.

### BRIEF SUMMARY OF THE INVENTION

Initial asexual reproduction of ‘PACthree’ by cuttings was first done in Santa Barbara, Calif., USA. The reproduction was conducted in controlled greenhouse environments. This initial, and other subsequent propagations conducted in controlled environments have demonstrated that the characteristics of ‘PACthree’ are true to type and are transmitted from one generation to the next.

‘PACthree’ is a low and compact miniature rose with good vigor.

The objective of the hybridization of this rose variety for commercial greenhouse culture was to create a new and distinct variety with:

1. Uniform and abundant flowers with good keepability;

2. Attractive long lasting foliage and compact growth;

3. Year round flowering under glasshouse conditions;

4. Suitability for production from softwood cuttings in pots;

5. Durable flowers and foliage which make the variety suitable for distribution in the floral industry.

This combination of qualities was not present in previously available commercial cultivars of this type and distinguish ‘PACthree’ from all other varieties of which I am aware. The seeds from hybridization were planted in a controlled environment and evaluations were conducted on the resulting plants. ‘PACthree’ was selected by, Daniel Jauchen, in his development program in Santa Barbara, Calif., USA.

### BRIEF DESCRIPTION OF THE DRAWINGS

The accompanying color illustrations show as true as is reasonably to obtain in color photographs of this type, the typical characteristics of the buds, flowers, leaves, stems of ‘PACthree’. Specifically illustrated in:

### Photo Sheet 1:

1: Young shoot.

2: Bud before opening the sepals.

3: Bud at the stage of opening the sepals.

4: Bud at the stage of opening the petals.

5: Flower during course of opening.

6: Open flower—plan view—obverse.

7: Open flower—plan view—reverse.

8: Fully open flower—plan view—obverse.

9: Fully open flower—plan view—reverse.

### Photo Sheet 2:

10: Receptacle showing stamens and pistils.

11: Receptacle showing pistils (stamens and sepals removed).

12: Flower petals, detached—inner surface.

13: Flower petals, detached—outer surface.

14: Bare stem exhibiting thorns and flower attachment.

15: Three leaflets upper side.

16: Three leaflets reverse side.

17: Five leaflets upper side.

18: Five leaflets reverse side.

### DETAILED BOTANICAL DESCRIPTION OF THE VARIETY

The following is a detailed description of the Miniature Rose: Rosa hybrida ‘PACthree’.

The following observations, measurements, values and comparisons describe plants grown in glass houses in Santa Barbara, Calif., USA.

The age of the observed plants where 11 to 13 weeks after propagation by cuttings, and produced as flowering pot plants in container of 10 centimeter in diameter.

Environmental conditions in the cultivation area for the observed plants, has been temperatures from 64 to 78 degree Fahrenheit, and 10 to 20 hours day length with assimilation light. Light levels from 600 foot candle to 6000 foot candle. The relative humidity has been ranging from 50 to 85%.

Color references are made using The Royal Horticultural Society (London, England) Colour Chart, 1995, except where common terms of color are used. For a comparison, the nearest existing rose variety is ‘Ruiskjol’, a rose variety described and illustrated in U.S. Plant Pat. No. 12,672.

Chart 1 details several physical characteristics of ‘PACthree’ and ‘Ruiskjol’.

Chart 1:

| | ‘PACthree’ | ‘Ruiskol’ |
|---|---|---|
| Petal color, Upper | Red Group | Red Group |
| surface | 46A | 45A |
| Petal color, Reverse | Red Group | Red Group |
| surface | 46B | 46A |
| Petal count | 30-35 | 36 |

Parents: Unnamed seedling times unnamed seedling.

### Classification

Botanical: Rosa hybrida.
Commercial: Miniature.

### Plant

Plant growth: Moderately vigorous. Grows compact upright to bushy. When grown as 10 cm pot plant, the average height of the plant itself is 18 to 20 cm, and average width is 20 cm. When grown as a 15 cm pot plant, the average height of the plant itself is 22 cm to 27 cm, and average width is 30 cm. Production time is generally 11 to 13 weeks depending on average temperature, light level, and cultural practices.

### Stem

Color: Young wood: Yellow-Green Group 147C. Older wood: Yellow-Green Group 147B.
Thorns:
   Incidence.—3-4 thorns per stem.
   Size.—1 mm to 1.5 mm.
   Color.—Greyed-Purple Group 186D.
   Shape.—Deep concave.
Surface:
   Young wood: Smooth.
   Older wood: Smooth.
Stem diameter: 2 mm to 3 mm.
Internode length: 20 mm to 30 mm.
Numbers of internodes: 5 to 6.
Plant foliage: Leaves arranged alternately, compound with 3 to 7 leaflets per leaf, generally symmetrical, abundant, and flat in aspect. Stipules at petiole base.
Quantity of leaves: 5 to 6 per lateral branch.
Leaf size: Medium 60 mm-75 mm (length). times. 40-50mm (width), for 5 leaflet.
Petioles:
   Color.—Yellow-Green Group 148C-D, with intonations of Greyed-Purple Group 183C Margins: Entire Length: 9 mm to 11 mm Diameter: about 0.5 mm to 1 mm.
Stipules: Size: 5 mm to 8 mm(length) 1-2 mm(width) Surface: Smooth Color: Yellow-Green Group 147A-B, Margins: Serrated single.
Rachis: Color: Yellow-Green Group 148C-D, with intonations of Greyed-Purple Group 183C Margins: Entire Length: 16 mm to 25 mm Diameter: 0.5 mm to 1 mm.
Leaflets:
   Edge.—Serrated.
   Serration.—Single.
   Shape.—Ovate with acute apex and obtuse base.
   Texture.—Smooth.
   Appearance.—Dull.
   Size.—length: 15 mm to 35 mm. Width: 10 mm to 15 mm.
   Color.—Young foliage: Upper surface: Yellow-Green Group 147B Lower surface: Yellow-Green Group 147C, With intonations of Greyed-Red 182D.
Color: Mature foliage: Upper surface: Yellow-Green Group 147A Lower surface: Greyed-Green Group 191A.
Leaf vein color: Upper surface: Yellow-Green Group 147A with intonations of Greyed-Purple Group 183C. Reverse surface: Greyed-Green Group 191A.

### Inflorescence

Blooming habit: Recurrent.
Number of flowers: Generally 1 to 3 bud per flowering stem.
Peduncle:
   Color.—Yellow-Green Group 147B-C.
   Texture.—Smooth.
   Length.—30 mm to 40 mm Diameter: 1 mm to 2 mm.
   Strength.—Upright.
Receptacle: Surface: Smooth, glabrous.
   Shape.—Funnel-shaped.
   Size.—height: 5 mm to 6 mm width: 6 mm to 8 mm.
   Color.—Yellow-Green Group 147B.
Sepals:
   Quantity.—5.
   Shape.—Narrowly Ovate w. acute tip.
   Texture.—Leathery.
   Margin.—Foliaceous appendages on three of the five sepals.
   Appearance.—Dull.
   Color.—Upper surface: Yellow-Green Group 147A-B Reverse surface: Greyed-Green Group 190A.
   Size.—16 mm (length), 5 mm (width).
Buds:
   Size.—15 mm to 17 mm (high) 15 mm to 26 mm (width) upon opening.
   Shape.—Ovate.
   Color.—at ¼ opening, Red Group 46A.
Flower:
   Duration.—As a pot plant, flowers last from 16 to 21 days.
   Fragrance. None.
   Size.—45 mm to 50 mm in diameter. Depth: 18 mm to 22 mm.
   Form.—Shape of flower when viewed from the side. Up on opening: Pointed to cupped Open flower: Flat.
   Color.—Petals, upon opening: Upper surface: Red Group 46A Group Base in White Group 155C Reverse surface:. Red Group 46A Group Base in White Group 155C. Petals after opening: Upper surface: Red Group 46A Group Base in White Group 155C Reverse surface: Red Group 46A Group Base in White Group 155C.
   General tonality.—On Open flower: Third day: Red Group 46A Group Afterwards: Red Group 46A Group.
Petals:
Petal reflex: Outermost petals reflex backwards at opening . Fully open all petals reflex backwards.
Texture: Smooth.
Petal edge: Entire.
Petal count: Approximately 30 on the average per flower.
Petal size: Length 12 mm to 20 mm Width: 12 mm to 20 mm.
Shape: Outer petals: Round — ovate Inner petals: Ovate Apex: Orbicular, Base: Obtuse to rotundate.
Reproductive organs:
Stamen number: Approximately 45 on average per flower.
Stamens length: 4 mm to 5 mm.
Pollen: Color: Yellow-Orange Group 19D, Abundance: high.
Anthers: Size: 1-2 mm Color: Orange-White Group 159D, Shape: Oblong.
Filaments: Size: 3 mm to 4 mm.
Color: Orange-White Group 159D with intonations of Greyed-Purple Group 186A.
Pistils number: Approximately 20-25 on average per flower.
Stigmas: Location: Lower in location to anthers Color: Orange-White Group 159B.
Styles: Color: White Group 155B. Length: 1 mm to 2 mm.

### Development

Vegetation: Dense.
Blooming: Abundant.
Aptitude to bear fruit: Poor.
Resistance to diseases: Above average resistance to mildew and Botrytis under normal growing conditions in Santa Barbara, Calif., USA.
Hips/seeds has not been observed due to that the plant has never been grown to the stage of seed development, due to the fact, that the variety is developed for use as a flowering pot plant only.
Winter hardiness & drought/heat tolerance: Due to the fact, that this variety is a potted flowering plant, developed indoor use only, the plant is not tested for winter hardiness or drought/heat tolerance.

I claim

1. A new and distinct variety of rose plant of the miniature class, substantially as herein illustrated and described as a distinct and novel rose variety due to its abundant red flowers, attractive long lasting foliage, vigorous and compact growth, year round flowering under glasshouse conditions, suitability for production from softwood cuttings in pots, and durable flowers and foliage which make the variety suitable for distribution in the floral industry.
